(19) United States
(12) Patent Application Publication
(10) Pub. No.: US 2016/0151403 A1
(43) Pub. Date: Jun. 2, 2016

(54) SMALL MOLECULE MODULATORS OF P53 FAMILY SIGNALING

(71) Applicant: The Trustees of the University of Pennsylvania, Philadelphia, PA (US)

(72) Inventor: Wafik S. EL-DEIRY, Philadephia, PA (US)

(21) Appl. No.: 14/557,772

(22) Filed: Dec. 2, 2014

Related U.S. Application Data
(62) Division of application No. 12/306,173, filed on Sep. 14, 2009, now abandoned.
(60) Provisional application No. 60/815,153, filed on Jun. 20, 2006.
(60) Provisional application No. 60/831,203, filed on Jul. 17, 2006.

(51) Int. Cl. A61K 31/7048; A61K 31/437; A61K 31/704

(52) U.S. Cl. CPC A61K 31/7048; A61K 31/704; A61K 31/437

(57) ABSTRACT

This invention relates to methods for identifying compound capable of activating p53-responsive transcriptional activity in a p53-deficient tumor cell and the use of these compounds.

### CROSS-REFERENCE TO RELATED APPLICATIONS

This application is a divisional application of U.S. patent application Ser. No. 12/306,173, filed Sep. 14, 2009, which is a National Phase Application of PCT International Application No. PCT/US2007/014366, International Filing Date Jun. 20, 2007, claiming priority of U.S. Provisional Patent Applications, 60/815,153, filed Jun. 20, 2006, and 60/831,203, filed Jul. 17, 2006, all which are incorporated herein by reference in their entirety.

### FIELD OF INVENTION

This invention is directed to methods for identifying compound capable of activating p53-responsive transcriptional activity in a p53-deficient tumor cell and the use of these compounds.

### BACKGROUND OF THE INVENTION

p53 represents an important target for drug development because it provides a key difference between normal cells and tumor cells. p53 is mutated in over half of all human tumors and, among almost all the remaining tumors, the pathway of p53-induced cell cycle arrest and apoptosis is deficient due to MDM2 overexpression or ARF deficiency. Furthermore, deficiency of p53 activity in tumor cells promotes resistance to chemo- and radio-therapies and a more malignant phenotype. p53 also plays an important role in receptor-mediated extrinsic cell death, e.g. TRAIL-resistant bax-null cells can be sensitized to TRAIL by activation of p53 by chemotherapeutics. Efforts have been made to target p53 with an attempt to restore p53 function in tumor cells. These strategies include introduction of wild-type p53 into tumor cells and rescue of mutant p53 in a wild-type conformation, which led to the discovery of potent small molecules such as CP-31398 or PRIMA1. Efforts have also been directed at liberating wild-type p53 from blockade by MDM2 using small molecules such as the nutlins. However, strategies targeting p53-activated transcriptional responses or p53 family member expression in p53-deficient tumors have yet to be explored or described. In the absence of p53 or in the presence of mutant p53, p53 family members, e.g. p73, may function instead of p53 in the pathway of tumor suppression. It has been shown that p′73 can be activated by some chemotherapeutics and plays a role in DNA damage-induced cell cycle arrest and apoptosis.

There is therefore a need to identify more potent p53 stimulators capable of either destabilizing mutated endogenous p53 tumor suppressor, or activating genes associated with the normal function of p53.

### SUMMARY OF THE INVENTION

In one embodiment, the invention provides a method of testing a compound for ability to: activate p53-responsive transcriptional activity in a p53-deficient tumor cell, activating a gene or micro RNA acting as a tumor suppressor, a gene or micro RNA suppressing cell growth, a gene or micro RNA inducing cellular senescence, a gene or micro RNA inducing apoptosis or their combination; comprising the step of: stably transfecting a human p53 reporter gene into a tumor cell, wherein the reporter gene is operably linked to a bioluminescent gene reporter; contacting the luciferase expressing cell with a candidate compound; and using a non-invasive real-time imaging to detect expression of said luciferase, analyzing the ability of the compound to activate p53-responsive transcriptional activity.

In another embodiment, the invention provides a method of activating p53-responsive transcriptional activity in a p53-deficient tumor cell, comprising the step of contacting the tumor cell with a compound capable of activating the expression or function of p73, Rb, VHL, APC, GSK3-β, ATM, ATR, Chk1, Chk2, CHFR, FHIT, PTEN, IκB-α, Mxi1, p21, p27, p16, ARF, REDD1, DR5, or their combination.

In one embodiment, the invention provides a method of inducing apoptosis, or cell-cycle arrest, or both in a p53-deficient tumor cell, comprising the step of contacting the p53-deficient tumor cell with a compound capable of inducing expression of p21, KILLER/DR5, Bax, Bak, Bid, Puma, Noxa, Bnip3L, Bnip3, PIDD, Fas/APO1, caspase 8, caspase 9, caspase 10, caspase 3, caspase 6, caspase 7, APAF1, Smac/DIABLO, cytochrome c, FADD, TRAIL, Fas ligand, Bim, DR4 or their combination.

In another embodiment, the invention provides a method of inhibiting a p53-deficient adenocarcinoma in a subject, comprising the step of administering to the subject a therapeutically effective amount of a composition comprising a compound capable of activating p53-responsive transcriptional activity thereby inducing apoptosis, cell-cycle arrest or both in the p53-deficient tumor cell.

In one embodiment, provided herein is a method of testing a compound for ability to: activate a transcriptional activity in a tumor cell; activating a gene or micro RNA acting as a tumor suppressor; activate a gene or micro RNA suppressing cell growth; activate a gene or micro RNA inducing cellular senescence; activate a gene or micro RNA inducing apoptosis; or their combination; comprising the steps of: stably transfecting a reporter gene into a tumor cell, wherein the tumor cell is deficient in the gene or miRNA sought to be activated and wherein the reporter gene is operably linked to a detectable label and corresponds to the transcriptional activity, a tumor suppressor gene, a cell growth suppressor gene, a gene inducing cell senescence, a gene inducing apoptosis, or their combination; contacting the trasfected tumor cell with a candidate compound; and using a non-invasive real-time imaging to detect said label, analyzing the ability of the compound.

In another embodiment, provided herein is a method of testing a compound for ability to modulate an oncogenic pathway; comprising the steps of: stably transfecting a reporter gene into a tumor cell, wherein the tumor cell expresses an oncogenic gene or miRNA sought to be activated and wherein the reporter gene corresponds to the oncogenic gene and is operably linked to a detectable label; contacting the transfected tumor cell with a candidate compound; and using a non-invasive real-time imaging to detect expression of said luciferase, analyzing the ability of the compound to modulate oncogenic activity.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIGS. 1A-1B show functional screening of the NCI DTP diversity set for p53-family transcriptional activators in SW480 mutant p53-expressing human colon cancer cells. A. SW480 cells, stably expressing the p53-responsive reporter PG13-luc were seeded in 96-well plates at a density of 5×10⁴ cells/well. p53-responsive transcriptional activity was imaged by the IVIS imaging system after exposure to the diversity set. B. Secondary screening with selected compounds at 2-fold increasing concentrations (range of 1-200 μM) and time points (as indicated);

FIGS. 2A-2B show protein levels of p53 target genes p21 and DR5 were induced by selected compounds in HCT/p53(+/+) cells (A) or HCT/p53(−/−) cells (B). In A, doses of compounds (μM) are listed above each lane. Cells treated with compounds were harvested and lysed for SDS-PAGE and immunoblotted with p21 or DR5 antibodies. Ran was used as a protein loading control. Doses of compounds in B were as follows: 2 μM for #15, 12 μM for #1 and #23, 20 μM for #20 and #32, 100 μM for #33, 200 μM for #5, #8, #12, #16, #17, and #22, and 400 μM for #3. The dose for adriamycin was 0.2 μg/ml. Cells were incubated for 16 hours at 37° C. with the various drugs prior to cell harvest;

FIGS. 3A-3F show: Structures (A) of isolated compounds and summary of their effects on the p53 family and transcriptional targets (B). Cell cycle profiles of HCT/p53(+/+) and HCT/p53(−/−) in response to treatment by selected compounds (C-E). The dose of #17 was 200 μM and for #23 it was 10 μM. F, p73 protein levels were elevated in HCT116/p53(−/−) cells in response to treatment by selected compounds at various concentrations as indicated;

FIG. 4 shows in-vivo anti-tumor effects of selected compounds. Balb/c nude mice were inoculated subcutaneously with 2 million HCT116/p53(−/−) cells in Matrigel on each flank. 6 mice were used in each group, in each of the two experiments. When the tumor mass reached about 3-5 mm, mice were treated with the compounds alone or following a single dose of TRAIL at 100 μg/mouse in experiment 1. At 7 days after treatment, mice were sacrificed and the tumor masses were weighted. The doses used were 100 mg/kg for #1, 50 mg/kg for #14, and 10 mg/kg for #23;

FIGS. 5A-5D show p53 transcriptional activity is induced in DLD1 xenografts and effects of knockdown of p73 by siRNA on drug-induced transcriptional activity. DLD1/PG13 cells were inoculated subcutaneously with 5 million cells. At 24 hours later mice were treated with selected compounds (100 mg/kg for #1, 50 mg/kg for #14 and #17, and 10 mg/kg for #23), and subsequently bioluminescence imaging was carried out after 16 hours. Two weeks later, tumor masses were weighed. A, bioluminescence imaging of p53 transcriptional activity induced in vivo. B, calculated fold-induction of p53 transcriptional activity. C, inhibition of tumor growth by selected compounds. D, effects of si-TAp73 on the transcriptional activity induced by selected compounds;

FIG. 6 shows mRNA levels of p53, DR5, and p21 in HCT116 cells after treatment by the indicated compounds;

FIGS. 7A-7C show: p53 levels and posttranslational modifications after treatment by selected compounds. (A) HCT116/p53(+/+) cells were treated with adriamycin, compounds nos. 1, 14, 17, and 23 for 16 h, and p53 and acetylated p53 levels were detected by Western blot. Nonspecific bands (Lower) serve as a loading control. (B) HCT116/p53(+/+)/PG13 cells were treated with adriamycin or compounds as indicated for 16 h and p53, phosphorylated p53 (ser20) levels and firefly luciferase protein expression were detected by Western blot. (C) SW480 cells were treated with compounds as indicated for 16 h, and p53 and phosphorylated p53 (ser20) levels were detected by Western blot. CP, CP-31398;

FIG. 8 shows p53 transcriptional activity induced in HCT116/p53(+/+)/PG-13 and HCT116/p53(−/−)/PG13 cells by selected compounds. Cells were treated by the indicated compounds at progressive 1:2 serial dilutions for 16 h, and the luciferase activities were detected by bioluminescence imaging. Fold induction was calculated by comparing to the nontreated cells;

FIG. 9 shows p53-responsive transcriptional activity induced in p53-mutant DLD1 cells and p53-null SKOV3 cells after treatment by selected compounds. C, nontreated control; and

FIGS. 10A-10B show knockdown of p73 protein expression by siRNA. HCT116/p53(−/−) cells were infected with retrovirus expressing siRNA targeting human p′73 and selected with blasticidine. Cells were treated with CPT-11 for 16 h and immunoblotted with p73 antibody.

### DETAILED DESCRIPTION OF THE INVENTION

This invention relates in one embodiment to methods for identifying compound capable of activating p53-responsive transcriptional activity in a p53-deficient tumor cell and the use of these compounds.

In one embodiment, a chemical library screen is performed using the methods described hereinbelow, by a strategy using bioluminescence imaging to identify small molecules that can induce a p53-responsive transcriptional activity and subsequent apoptosis in tumor cells deficient in p53. In another embodiment, the methods provided herein, comprising the use of bioluminescence imaging to screen for potential p53 activators has advantages over other conventional methods because it is sensitive and non-invasive and it allows the recording of real-time kinetics of transcriptional change over a time period up to 2-3 days.

Accordingly and in one embodiment, provided herein is a method of testing a compound for ability to activate p53-responsive transcriptional activity in a p53-deficient tumor cell, comprising the step of: stably transfecting a human p53 reporter gene into a tumor cell, wherein the reporter gene is operably linked to a firefly luciferase protein; contacting the luciferase expressing cell with a candidate compound; and using a non-invasive real-time imaging to detect expression of said luciferase, analyzing the ability of the compound to activate p53-responsive transcriptional activity. In one embodiment, the bioluminescent reporter used in the methods described herein, is green fluorescence protein (GFP).

In one embodiment, aminoluciferin is operably linked to DEVD (benzyloxyycarbonyl aspartyl glutamylvalylaspartic acid fluoromethyl ketone), VEHD (benzyloxyycarbonyl valyl glutamyl histidylaspartic acid fluromethyl ketone), LETD (benzyloxycarbonyl leucylglutamylthreonylaspartic acid fluoromethyl ketone), LEHD (benzyloxycarbonyl leucylglutamylhistidylaspartic acid fluoromethyl ketone), IEPD (benzyloxycarbonyl Isoleucylglutamylprolylaspartic acid fluoromethyl ketone), DETD (benzyloxycarbonyl aspartylglutamylthreonylaspartic acid fluoromethyl ketone), WEHD (tryptophyl glutamylhistidylaspartic acid fluromethyl ketone), YVAD (benzyloxycarbonyl tyrosylvalylalanyl aspartic acid fluoromethyl ketone), VEID (benzyloxycarbonyl valylglutamyl isoleucylaspartic acid fluoromethyl ketone). “Operatively linked” refers in one embodiment to a juxtaposition wherein the components so described are in a relationship permitting them to function in their intended manner. In one embodiment aminoluciderin is “operably linked” to DEVD, acting as a substrate for caspase-7, which is involved in apoptosis in one embodiment and whose action results in the release of aminoluciferin from DEVD, thereby making it accessible to react with luciferase.

In another embodiment, provided herein is a method of testing a compound for ability to: activate p53-responsive transcriptional activity in a p53-deficient tumor cell, activating a gene or micro RNA acting as a tumor suppressor, a gene or micro RNA suppressing cell growth, a gene or micro RNA inducing cellular senescence, a gene or micro RNA inducing apoptosis or their combination; comprising the step of: stably transfecting a human p53 reporter gene into a tumor cell, wherein the reporter gene is operably linked to a bioluminescent gene reporter; contacting the luciferase expressing cell with a candidate compound; and using a non-invasive real-time imaging to detect expression of said luciferase, analyzing the ability of the compound to activate p53-responsive transcriptional activity.

In one embodiment, provided herein is a method of testing a compound for ability to: activate p53-responsive transcriptional activity in a p53-deficient tumor cell, activating a gene or micro RNA acting as a tumor suppressor, a gene or micro RNA suppressing cell growth, a gene or micro RNA inducing cellular senescence, a gene or micro RNA inducing apoptosis or their combination; comprising the step of: stably transfecting a human p53 reporter gene into a tumor cell, wherein the reporter gene is detectably labeled; contacting the detectably labeled tumor cell with a candidate compound; and using a non-invasive real-time imaging to detect said label, analyzing the ability of the compound to activate p53-responsive transcriptional activity. In one embodiment, the human p53 reporter gene is detectably labeled with a luminescent agent, a detectable cell marker. In another embodiment, analysis is done on product of genes activated using the compounds described herein.

In another embodiment, provided herein is a method of testing a compound for ability to: activate a transcriptional activity in a tumor cell; activating a gene or micro RNA acting as a tumor suppressor; activate a gene or micro RNA suppressing cell growth; activate a gene or micro RNA inducing cellular senescence; activate a gene or micro RNA inducing apoptosis; or their combination; comprising the steps of: stably transfecting a reporter gene into a tumor cell, wherein the tumor cell is deficient in the gene or miRNA sought to be activated and wherein the reporter gene is operably linked to a detectable label and corresponds to the transcriptional activity, a tumor suppressor gene, a cell growth suppressor gene, a gene inducing cell senescence, a gene inducing apoptosis, or their combination; contacting the transfected tumor cell with a candidate compound; and using a non-invasive real-time imaging to detect said label, analyzing the ability of the compound.

In another embodiment, provided herein is a method of testing a compound for ability to modulate an oncogenic pathway; comprising the steps of: stably transfecting a reporter gene into a tumor cell, wherein the tumor cell expresses an oncogenic gene or miRNA sought to be activated and wherein the reporter gene corresponds to the oncogenic gene and is operably linked to a detectable label; contacting the transfected tumor cell with a candidate compound; and using a non-invasive real-time imaging to detect expression of said luciferase, analyzing the ability of the compound to modulate oncogenic activity.

In one embodiment, the methods of testing provided herein, further comprise a validation step. In one embodiment, the validation step comprises grafting the transfected tumor cell onto a subject; contacting the subject with the test compound; and analyzing the capability of the compound to modulate in one embodiment, or activate, inhibit, increase, suppress, arrest or a combination thereof, of the activity sought, thereby validating the compounds therapeutic capability.

In one embodiment, the term “detectably labeled” refers to any detectable tag that can be attached directly (e.g., a fluorescent molecule integrated into a polypeptide or nucleic acid) or indirectly (e.g., by way of activation or binding to an expressed genetic reporter, including activatable substrates, peptides, receptor fusion proteins, primary antibody, or a secondary antibody with an integrated tag) to the molecule of interest. In another embodiment, the term “detectably labeled” refers to any tag that can be visualized with imaging methods. The detectable tag can be a radio-opaque substance, radiolabel, a fluorescent label, a light emitting protein, a magnetic label, or microbubbles (air filled bubbles of uniform size that remain in the circulatory system and are detectable by ultrasonography, as described in Ellega et al. Circulation, 108:336-341, 2003, which is herein incorporated in its entirety). The detectable tag can be gamma-emitters, beta-emitters, and alpha-emitters, positron-emitters, X-ray-emitters, ultrasound reflectors (microbubbles), or fluorescence-emitters suitable for localization. Suitable fluorescent compounds include fluorescein sodium, fluorescein isothiocyanate, phycoerythrin, Green Fluorescent Protein (GFP), Red Fluorescent Protein (RFP), Texas Red sulfonyl chloride, as well as compounds that are fluorescent in the near infrared such as Cy5.5, Cy7, and others. In another embodiment, the term “detectably labeled” refers to genetic reporters detectable following administration of radiotracers such as hSSTr2, thymidine kinase (from herpes virus, human mitochondria, or other) and NIS (iodide symporter). In another embodiment, the term “detectably labeled” refers to Light emitting proteins such as, in certain embodiments; various types of luciferase. Those skilled in the art will know, or will be able to ascertain with no more than routine experimentation, other fluorescent compounds that are suitable for labeling the reporter compounds described and used in the methods provided herein.

As used herein, the term “cell surface markers” refers in one embodiment, to a gene or peptide expressed by the gene whose expression level, alone or in combination with other genes, is correlated with the presence of tumorigenic cancer cells. The correlation may relate to either an increased or decreased expression of the gene (e.g. increased or decreased levels of mRNA or the peptide encoded by the gene), or its encoded proteins. In one embodiment, the cell marker is CD4, or a growth hormone, macrophage-inhibitory factor, TRAIL, or their combination in other embodiments. In one embodiment, the cell marker is CD4, or CD44, SC-1, Fas/AP0-1/CD95, bcl-2, Ki-67, CD34 and the like in other embodiments.

In one embodiment, the term “operably linked” refers to the linkage of nucleic acid sequences in such a manner that a nucleic acid molecule capable of directing the transcription of a given gene and/or the synthesis of a desired protein molecule is produced. In another embodiment, the term “operably linked” refers to the linkage of amino acid sequences in such a manner so that a functional protein is produced, or in another embodiment, maintained. “Operably linked” is defined in another embodiment, as the expression of a nucleic acid under the control of a given promoter sequence; i.e., the promoter controls the expression of a given nucleic acid. The given nucleic acid can be, but is not limited to, a reporter nucleic acid.

In another embodiment, the term “gene expression” refers to the process of converting genetic information encoded in a gene into RNA (e.g., mRNA, rRNA, tRNA, or snRNA) through “transcription” of the gene such as in another embodiment, via the enzymatic action of an RNA polymerase and for protein encoding genes, into protein through “translation” of mRNA. Gene expression can be regulated at many stages in the process. In one embodiment, the terms “Upregulation” or “activation” refer to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively in other embodiments.

In one embodiment, the term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

In one embodiment, transcriptional control signals in eukaryotes comprise “promoter” and “enhancer” elements. In another embodiment, promoters and enhancers consist of short arrays of DNA sequences that interact specifically with cellular proteins involved in transcription. The selection of a particular promoter and enhancer depends in one embodiment, on what cell type is to be used to express the protein of interest. Some eukaryotic promoters and enhancers have a broad host range while others are functional in a limited subset of cell types.

In another embodiment, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

In one embodiment, the process and methods of screening a compound capable of molecular target modulation by activating p53-responsive transcriptional activity in a p53-deficient tumor cell, comprising the step of: stably expressing a human p53 reporter gene in a tumor cell or in another embodiment, the promoter is capable of activating gene expression of any tumor suppressor gene such as p73 in one embodiment, or Rb, VHL, APC, GSK3-β, ATM, ATR, Chk1, Chk2, CHFR, FHIT, PTEN, IkB-α, Mxi1, p21, p27, p16, ARF, REDD1 in other embodiment, or in one embodiment, any gene or micro-RNA that can suppress cell growth, or in another embodiment, induce cellular senescence in another embodiment, induce apoptosis such as KILLER/DR5 in one embodiment, or Bax, Bak, Bid, Puma, Noxa, Bnip3L, Bnip3, PIDD, Fas/APO1, caspase 8, caspase 9, caspase 10, caspase 3, caspase 6, caspase 7, APAF1, Smac/DIABLO, cytochrome c, FADD, TRAIL, Fas ligand, Bim, DR4 in other embodiment, wherein the reporter gene is operably linked to a firefly luciferase expressing gene in another embodiment or other fluorescent or bioluminescent gene reporter. In one embodiment; the luciferase or other reporter-expressing cell is contacted with a candidate small molecule compound, or synthetic peptide, synthetic oligonucleotide, micro-RNA, polypeptide or antibody in other embodiment; and using a non-invasive real-time imaging, analyzing the ability of the compound to activate p53-responsive transcriptional or other growth inhibitory or apoptosis-inducing gene promoter activity is tested using the methods described herein.

In one embodiment, a coupling activity of the molecular target modulation, results in high luciferase activity due to activation of a p53 or p53-like transcriptional activity or in another embodiment, any other specific tumor suppressive, specific growth inhibitory, specific senescence-inducing or specific apoptosis-inducing promoter linked to a cellular bioluminescent or fluorescent reporter activity for real-time imaging with an actual growth inhibitory or cell death response as can be imaged as a function of time or increasing dose of small molecule, peptide or antibody. In another embodiment, the coupling activity allows to have anti-tumor effects during the screening phase of the identification of those candidate lead compounds. The coupling of molecular target modulation with growth inhibition in one embodiment, or cell elimination or cell death induction in other embodiments; on multi-well plates in a cell-based assay provides an extremely efficient and novel method to accelerate the identification of agents (e.g. small molecule compounds, peptides, oligonucleotides, micro-RNAs, polypeptides or antibodies), predicted to have anti-tumor effects. In another embodiment, seamless transition to an in vivo validation of molecular target activation is effected using the methods provided herein. This part of the process provides in another embodiment, a method of immediately observing the use of non-invasive imaging of the activation of p53 of molecular target using xenografted tumor cells that in another embodiment, carry the promoter-driven reporter. Treatment of mice or other subjects carrying the genetically modified human tumor reporter-carrying xenografts with therapeutic agents provides an efficient method as part of the screening-validation process to verify molecular target modulation in vivo. In one embodiment, using a dual reporter, such as Firefly luciferase to report on molecular target modulation and renilla luciferase to report on tumor volume, allows in certain embodiments, for a second coupling of molecular target modulation and in vivo anti-tumor effects in tumor xenograft-bearing subjects. In one embodiment, molecular target validation in vivo using gene silencing of the molecular target is carried out. In one such embodiment, a small molecule that restores p53 transcriptional activity and reporter gene activation in a p53-deficient cell through stimulation of p73, is expected to lose this activity in tumor cells carrying shRNA or other genetic or dominant-negative inhibitors of p73 expression. In one embodiment, Structure/Activity Relationship by Imaging (SAR-by-Imaging) is an important part of the screening-validation and development process effected using the methods provided herein, by providing a visual method to identify agents (small molecules, peptides, oligonucleotides, micro-RNAs, polypeptides, or antibodies) that effectively modulate the molecular target at much lower doses that may provide favorable pharmacokinetic or pharmacodynamic properties as well as a much higher therapeutic window. Thus in addition to the original screen design, and in another embodiment, the steps of the screening-validation and development process described herein, provide a novel seamlessly connected efficient method to accelerate the lengthy preclinical phase of drug discovery and development.

In one embodiment, the term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. In another embodiment, the term “stable transfection” or “stably transfected” refers to the introduction and integration of foreign DNA into the genome of the transfected cell. The term “stable transfectant” refers to a cell which has stably integrated foreign DNA into the genomic DNA.

In one embodiment, the term “target genes” refers to genes of any kind and origin the expression of which is regulated by p53. Embodiments of such genes are RGC, MCK, mdm2, cyclin G, synthetic p53 reporter genes, p21 and bax. In another embodiment, p21 is held responsible for the growth stand-still of the cell caused by p53 and bax is held responsible for the cell death caused by p53. In one embodiment, the expression “target genes” refers to the promoter sequences thereof and in another embodiment, p53 binding sequences thereof. In one embodiment, the target genes are present in any DNA conformation. They can be present in cells, particularly tumor cells in another embodiment, or occur in isolated fashion in certain embodiment of the methods described herein. In one embodiment, the target genes are present in connection with further sequences, particularly with those coding for a reporter protein, such as PG13-luc reporter gene in one embodiment.

In one embodiment, the methods provided herein are used to test compounds capable of activating transcription factors. In another embodiment, the transcription factors is NFκB, or HIF1-α, HIF2-α, Beta-catenin, c-Jun, AP1, or their combination in other discrete embodiments of each. In one embodiment, the tested compounds, once found effective are used to modulate the activity of the genes or miRNA provided herein.

In another embodiment, the methods provided herein are used to test compounds capable of activating a tumor suppressor gene. In one embodiment, the tumor suppressor gene is p73, or pRb, VHL, APC, GSK3-β, ATM, ATR, Chk1, Chk2, CHFR, FHIT, PTEN, IκB-α, Mxi1, p21, p27, p16, ARF, REDD1, or their combination in other discrete embodiments of each or any combination thereof.

In one embodiment, the methods provided herein are used to test compounds capable of activating genes or miRNA inducing apoptosis. In another embodiment, the gene or its encoded protein capable of inducing apoptosis is KILLER/DR5, or Bax, Bak, Bid, Puma, Noxa, Bnip3L, Bnip3, PIDD, Fas/APO1, caspase 8, caspase 9, caspase 10, caspase 3, caspase 6, caspase 7, APAF1, Smac/DIABLO, cytochrome c, FADD, TRAIL, Fas ligand, Bim, DR4 or their combination in other discrete embodiments of each or any combination thereof.

With the development of real-time non-invasive bioluminescent imaging of p53 transcriptional activity in vitro and in vivo, a high throughout cell-based functional screen for small molecules that trigger a p53-like transcriptional response in p53-deficient tumor cells becomes possible. In one embodiment, SW480 human adenocarcinoma cells that expressed a p53-responsive firefly luciferase reporter were exposed to the diversity set of small molecules collected by NCI. In another embodiment, structurally related as well as structurally dissimilar molecules are identified and used in the methods provided herein, which activate p53-responsive transcriptional activity in p53-deficient tumor cells. In another embodiment, the compounds described herein have a potent anti-tumor effects on HCT116/p53^{-/-} or DLD1 human tumor xenografts. In one embodiment, the methods of screening described hereinbelow, establish the feasibility of a cell-based drug screening strategy using bioluminescence to target the p53 transcription factor family in human cancer and in another embodiment, provide lead compounds for further development in cancer therapy.

The skilled person in the art would readily recognize that the methods provided herein, can be performed with any tumor cell line deficient in a tumor suppressor gene or carrying a deletion or mutation of a specific tumor suppressor gene. In another embodiment, the tissue of origin of the tumor cell can include colon, small intestine, stomach, liver, kidney, lung, skin, brain, breast, prostate, lymph node, lympoid, thymus, adrenal, thyroid, osteosarcoma, bladder, ovary, uterus, or bone.

In another embodiment, the non-invasive real-time imaging step, used in the methods described herein comprises; incubating the contacted luciferase-expressing tumor cells; and measuring luminescence intensities, wherein the higher the measured luminescent intensity, the higher is the degree of molecular target modulation. In one embodiment, the degree of modulation of the candidate compound is coupled as described in the method described herein with programmed cell death level, the cell-cycle arrest, or both as well as tumor cell elimination in some embodiments, through use of a dual reporter such as Firefly luciferase to report on molecular target modulation and renilla luciferase to report on tumor volume as described in an embodiment of the methods provided herein.

In one embodiment, the p53-deficient tumor cell used in the methods of testing a single compound, or in another embodiment, the high-throughput screening of many compounds as described herein, is a human colon adenocarcinoma cell. In one embodiment, the a human colon adenocarcinoma cell line is SW480 human adenocarcinoma cells that expressed a p53-responsive firefly luciferase reporter.

The tumor-suppressive function of p53 are attributed in one embodiment to its participation in the cellular response to DNA damage. In response to DNA strand breaks or transcription blocking DNA damage, such as UV light-induced photoproducts in one embodiment, p53 accumulates through a posttranscriptional mechanism. In another embodiment, p53 protein acts as an activator and as a repressor of transcription in another embodiment. In one embodiment, p53 transactivation function plays a role in the regulation of the G₁ and G₂ cell cycle checkpoints, or in another embodiment, the induction of apoptosis, and the stimulation of nucleotide excision repair (NER) in other embodiment.

In one embodiment, a single base substitution results in the synthesis of proteins having a different growth regulatory properties and, in another embodiment, lead to malignancies. In another embodiment, p53 promotes cell cycle arrest by transactivating critical target genes. In another embodiment, the genes activated are p21^{WAF1}; GADD45; and 14-3-3σ. In one embodiment, p21^{wAF1} protein p21, binds to and inactivates cyclin-dependent kinases, arrests cells in G₁ and prevents S-phase entry. In another embodiment, p53 target genes with proapoptotic activity fall into three groups based on their subcellular location. In one embodiment, the group of genes encode proteins localized to the cell membrane is KILLER/DR5. In one embodiment, KILLER/DR5 is a member of the tumor necrosis factor receptor superfamily that is induced by DNA damage in a p53-dependent manner and in another embodiment, is sufficient to induce apoptosis.

In one embodiment, the mutation in the p53-deficient tumor cell making the cell p53 deficient and is used in the methods of testing a compound for ability to activate p53-responsive transcriptional activity is R273H. In another embodiment, the mutation is P309S, or their combination in another embodiment. In another embodiment, the tumor cell may harbor deletion or mutation or a tumor suppressor, growth inhibitor, sensescence inducer or cell death inducing gene. In another embodiment, the tumor cell used for screening of compounds as described herein, also contain loss of heterozygosity of one allele of the tumor suppressor gene, senescence inducer, apoptosis-inducer, growth inhibitory gene or micro-RNA, either alone or in combination with a mutated (or hypermethylated) second allele leading to loss of gene function in the tumorigenic cells. In another embodiment, other tumor suppressor genes that could be involved in loss of heterozygosity of one allele and mutation or hypermethylation of the second allele include p73, Rb, VHL, APC, GSK3-beta, ATM, ATR, Chk1, Chk2, CHFR, FHIT, PTEN, IkB-alpha, Mxi1, p21, p27, p16, ARF, REDD1 or any gene or micro-RNA that can suppress cell growth, induce cellular senescence or induce apoptosis such as KILLER/DR5, Bax, Bak, Bid, Puma, Noxa, Bnip3L, Bnip3, PIDD, Fas/APO1, caspase 8, caspase 9, caspase 10, caspase 3, caspase 6, caspase 7, APAF1, Smac/DIABLO, cytochrome c, FADD, TRAIL, Fas ligand, Bim, DR4, or their combination in certain embodiments.

Caspase-3 and -7 are members of the cysteine aspartic acid-specific protease (caspase) family, which play an effector roles in apoptosis in mammalian cells. The results of cell lysis due to programmed cell death in another embodiment, is followed by caspase cleavage of the substrate and generation of a luminescent signal, produced by luciferase. In one embodiment, luminescence is proportional to the amount of caspase activity present and therefore to the extent of programmed cell death.

In another embodiment, the methods provided herein, for testing a compound for ability to activate p53-responsive transcriptional activity in a p53-deficient tumor cell, comprise a step of non-invasive real-time imaging. In one embodiment, the non-invasive real-time imaging comprises; incubating the contacted luciferase-expressing tumor cells; and measuring luminescence intensities, wherein the higher the measured luminescent intensity, the higher is the programmed cell death level, the cell-cycle arrest, or both. In one embodiment, the compound tested using the methods described herein, or in another embodiment, used in the compositions and certain methods described herein is a small molecule compound, a synthetic peptide, a synthetic oligonucleotide, a micro-RNA, a polypeptide or an antibody.

In one embodiment, the term “apoptosis inducing agents”, refer to compositions such as genes encoding the tumor necrosis factor related apoptosis inducing ligand termed TRAIL, and the TRAIL polypeptide (U.S. Pat. No. 5,763,223; incorporated herein by reference); the 24 kD apoptosis-associated protease of U.S. Pat. No. 5,605,826 (incorporated herein by reference); Fas-associated factor 1, FAFI (U.S. Pat. No. 5,750,653; incorporated herein by reference). Also contemplated for use in these aspects of the present invention is the provision of interleukin-1β-converting enzyme and family members, which are also reported to stimulate apoptosis.

In one embodiment, bioluminescence images are acquired with the charge-coupled device (CCD) camera and luminescence intensity is quantified using the Living Image software (version 2.5) from Xenogen. In one embodiment, the luminescence intensities measured, are those captured by the CCD camera, translated to arbitrary luminescence units (ALU). As used herein, higher luminescence refers to those captured bioluminescence images, exhibiting greater ALU values than a standard. In one embodiment, the measured bioluminescence of a cell before being contacted with apoptosis-inducing agent serves as bioluminescence standard and is designated an index ALU number.

In one embodiment, the increase in ALU following exposure to apoptosis-inducing agent reflects the degree of apoptosis or programmed cell death and therefore, the higher the measured luminescent intensity above and beyond the index ALU, the higher is the programmed cell death level.

In one embodiment, the term “antibody” include complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies of the invention which are useful for the compositions and methods described herein can be from any source, and in addition may be chimeric. In one embodiment, sources of antibodies can be from a mouse, or a rat, or a human in other embodiments. Antibodies of the invention which are useful for the compositions and methods of the invention have reduced antigenicity in humans, and in another embodiment, are not antigenic in humans. Chimeric antibodies as described herein contain in one embodiment, human amino acid sequences and include humanized antibodies which are non-human antibodies substituted with sequences of human origin to reduce or eliminate immunogenicity, but which retain the binding characteristics of the non-human antibody.

In one embodiment, the terms “Peptides,” “polypeptides” and “oligopeptides” refer to chains of amino acids (typically L-amino acids) in which carbons are linked through peptide bonds formed by a condensation reaction between the carboxyl group of the carbon of one amino acid and the amino group of the carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free amino group on the amino acid at the amino terminal of the peptide, or to the amino group (imino group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

“Nucleic acid,” as used herein, refers to a deoxyribonucleotide (DNA) or ribonucleotide (RNA) in either single- or double-stranded form, and unless otherwise limited, encompasses known analogs of natural nucleotides which can function in a manner similar to the naturally occurring nucleotides.

In another embodiment, the term “synthetic oligonucleotide” refers to chemically synthesized polymers of 12 to 50, or in another embodiment from about 15 to about 30, ribonucleotide and/or deoxyribonucleotide monomers connected together or linked by at least one or more than one, 5′ to 3′ internucleotide linkage. In another embodiment, the term “oligonucleotide” includes linear oligomers of nucleotides or derivatives thereof, including deoxyribonucleosides, ribonucleosides, and the like.

In another embodiment, the term “MicroRNAs” (miRNAs) refers to a class of gene products that repress mRNA translation or in one embodiment, mediate mRNA degradation in a sequence-specific manner in animals and plants. In one embodiment, the term “miRNA” is used interchangeably with artificial small noncoding RNA (ncRNAs). In another embodiment, the compounds tested using the methods described herein, or used in the compositions and methods described herein are used as therapeutics. In another embodiment, ncRNAs interfere with RNA transcription, stability, translation or directly hamper the function of the targets by binding to their surface.

In one embodiment, aminoluciferin represents a leaving group. The liberated aminoluciferin can be luminometrically detected even in smallest concentrations. in one embodiment, the liberated aminoluciferin is reacted with the enzyme luciferase of the firefly Photinus pyralis or of the firefly Photinus plathiophthalamus or of the luciferase of other species or chemically or genetically modified luciferases in the presence of ATP+MgCl₂. In the course of said reactions photons are emitted; i.e. in the course of the reaction with the enzyme of the firefly Photinus pyralis at 605 nm in one embodiment and in the course of the reaction with the enzyme of the firefly Photinus plathiophthalamus at 549 or 570 nm in another embodiment, or wavelength corresponding to the used luciferin/luciferase system, respectively. The emission at 549 nm takes place if the enzyme originates from the dorsal organ of the firefly mentioned whereas the emission at 570 nm takes place if the enzyme originates from the ventral organ.

A luciferase is an enzyme that catalyzes a reaction to produce light. There are a number of different luciferase enzymes derived or modified from various sources, including firefly luciferase in one embodiment, and Renilla luciferase in another embodiment. “Renilla luciferase” refers to a luciferase enzyme isolated from a member of the genus Renilla or an equivalent molecule obtained from any other source or synthetically.

In one embodiment, the term “cell death” includes the processes by which mammalian cells die. Such processes include apoptosis (both reversible and irreversible) and processes thought to involve apoptosis (e.g., cell senescence), as well as necrosis. “Cell death” is used in one embodiment to refer to the death or imminent death of nucleated cells (e.g., neurons, myocytes, hepatocytes and the like) as well as to the death or imminent death of anucleate cells (e.g., red blood cells, platelets, and the like). Cell death is typically manifested by the exposure of PS on the outer leaflet of the plasma membrane. Apoptosis refers in one embodiment to “programmed cell death” whereby the cell executes a “cell suicide” program. In another embodiment, the apoptosis program is evolutionarily conserved among virtually all multicellular organisms, as well as among all the cells in a particular organism. Further, it is believed that in many cases, apoptosis may be a “default” program that must be actively inhibited in healthy surviving cells. All apoptosis pathways appear to converge at a common effector pathway leading to proteolysis of key proteins. Caspases are involved in both the effector phase of the signaling pathway and further upstream at its initiation. The upstream caspases involved in initiation events become activated and in turn activate other caspases that are involved in the later phases of apoptosis.

In one embodiment, luminescence intensity measured in the methods described herein, is quantified using the Living Image software (version 2.5) from Xenogen.

In another embodiment, the step of incubating the contacted luciferase-expressing tumor cells in the methods described herein is done for between about 0 to about 84 hours. In another embodiment the incubation of the contacted luciferase-expressing tumor cells and the candidate test compound is done for for between about 6 to about 84 hours, or in another embodiment, between about 6 to about 12 hours, or in another embodiment, between about 12 to about 18 hours, or in another embodiment, between about 18 to about 24 hours, or in another embodiment, between about 24 to about 30 hours, or in another embodiment, between about 30 to about 36 hours, or in another embodiment, between about 36 to about 42 hours, or in another embodiment, between about 42 to about 48 hours, or in another embodiment, between about 48 to about 72 hours, or in another embodiment, between about 72 to about 84 hours.

In one embodiment, the compounds tested in the methods described hereinabove, are used in the methods provided herein. Accordingly and in another embodiment, provided herein is a method of activating p53-responsive transcriptional activity in a p53-deficient tumor cell, comprising the step of contacting the tumor cell with a compound capable of activating the expression or function of p21, DR5, p73, or their combination. In one embodiment, the compound capable of activating the expression or function of p21, DR5, p73, or their combination, is wild-type (WT) p53.

In another embodiment, provided herein is a method of activating p53-responsive transcriptional activity in a p53-deficient tumor cell, comprising the step of contacting the tumor cell with a compound capable of activating the expression or function of p73, Rb, VHL, APC, GSK3-β, ATM, ATR, Chk1, Chk2, CHFR, FHIT, PTEN, IκB-α, Mxi1, p21, p27, p16, ARF, REDD1, DR5, or their combination. In another embodiment, the human p53 reporter gene is operably linked to a bioluminescent compound, such as luciferase in one embodiment. In one embodiment, any human p53 reporter gene is operably linked to a bioluminescent compound described herein may be used in the methods described herein, such as dual reporter, such as Firefly luciferase to report on molecular target modulation and renilla luciferase to report on tumor volume.

In one embodiment, the compounds screened with the methods described herein, or in other embodiments, used in the compositions of the methods described herein restore functional wild-type gene and protein signaling in cells that in another embodiment, lost the specific signaling pathways contributing to tumor development through loss of heterozygosity in one embodiment, or gene mutation or hypermethylation- or micro-RNA-induced gene silencing in other embodiments.

In another embodiment, the compound capable of activating the expression or function of p21, DR5, p73, or their combination, is any one of the compounds of Table I, or their combination in another embodiment.

TABLE I
Thirty-three compounds screened from the
National Cancer Institute (NCI) diversity set

| No. | NSC no. | Mᵣ |
|---|---|---|
| 1 | 5159 | 641 |
| 2 | 13768 | 295 |
| 3 | 28992 | 192 |
| 4 | 45236 | 417 |
| 5 | 49692 | 279 |
| 6 | 94914 | 270 |
| 7 | 101824 | 262 |
| 8 | 105900 | 284 |
| 9 | 109816 | 306 |
| 10 | 117028 | 398 |
| 11 | 123111 | 331 |
| 12 | 127133 | 434 |
| 13 | 130796 | 392 |
| 14 | 143491 | 579 |
| 15 | 146109 | 317 |
| 16 | 150412 | 359 |
| 17 | 162908 | 326 |
| 18 | 169453 | 300 |
| 19 | 175650 | 366 |
| 20 | 176327 | 406 |
| 21 | 204936 | 257 |
| 22 | 211340 | 303 |
| 23 | 254681 | 563 |
| 24 | 295558 | 612 |
| 25 | 295642 | 399 |
| 26 | 306960 | 295 |
| 27 | 320656 | 225 |
| 28 | 338571 | 257 |
| 29 | 339585 | 340 |
| 30 | 371688 | 400 |
| 31 | 373529 | 632 |
| 32 | 407807 | 391 |
| 33 | 639174 | 457 |

NSC, National Service Center

In another embodiment, compounds used in the methods described herein, or identified by the methods provided herein are modified at various positions independently by addition of groups such as in one embodiment the group consisting of hydrogen, or alkyl, alkenyl, alkynyl, cycloalkyl, cycloalkenyl, aryl, heteroaryl, and heterocyclyl in other embodiments, wherein each of the R⁵ and R⁶ substituents alkyl, alkenyl, alkynyl, cycloalkyl, cycloalkenyl, aryl, heteroaryl and heterocyclyl substituents are optionally independently substituted by one to four moieties independently selected from halo, alkyl, alkenyl, alkynyl, perhaloalkyl, aryl, cycloalkyl, heteroaryl, heterocyclyl, formyl, —Cₙ, alkyl-(CO)—, aryl-(CO)—, HO—(CO)—, alkyl-O—(CO)—, H₂N—(CO)—, alkyl-NH—(CO)—, (alkyl)₂-N—(CO)—, aryl-NH—(CO)—, —NO₂, amino, alkylamino, (alkyl)₂-amino, alkyl-(CO)—NH—, aryl-(CO)—NH—, aryl-(CO)-[(alkyl)-N]—, H₂N—(CO)—NH—, alkyl-HN—(CO)—NH—, (alkyl)₂-N—(CO)—NH—, (alkyl)₂-N—(CO)-[(alkyl)-N]—, aryl-HN—(CO)—NH—, (aryl)₂-N—(CO)—NH—, (aryl)₂-N—(CO)-[(alkyl)-N]—, alkyl-O—(CO)—NH—, alkyl-O—NH—(CO)—, alkyl-O—NH—(CO)-alkyl-NH—(CO)—, aryl-O—(CO)—NH—, alkyl-S(O)₂NH—, aryl-S(O)₂NH—, alkyl-S—, alkyl-S(O)—, aryl-S(O)—, aryl-S—, hydroxy, alkoxy, perhaloalkoxy, aryloxy, alkyl-(CO)—O—, aryl-(CO)—O—, H₂N—(CO)—O—, alkyl-HN—(CO)—O—, (alkyl)₂-N—(CO)—O—, aryl-HN—(CO)—O— and (aryl)₂-N—(CO)—O—; wherein in other embodiments, when said cycloalkyl or aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring, which carbocyclic or heterocyclic ring is optionally fused to an aryl ring.

In another embodiment, the compounds described herein contain one or more asymmetric centers and thus give rise to enantiomers, diastereomers, and other stereoisomeric forms that may be defined, in terms of absolute stereochemistry, as (R)— or (S)—, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

In one embodiment, compound capable of activating the expression or function of p21, DR5, p73, or their combination, is NSC5159. In another embodiment, the compound is NSC143491. In another embodiment, the compound is NSC254681. In another embodiment, the compound is NSC639174, or their combination in certain other embodiments. In another embodiment, compound capable of activating the expression or function of p21, DR5, p73, or their combination, is NSC146505. In another embodiment, the compound is NSC160470. In another embodiment, the compound is NSC160471. In another embodiment, the compound is NSC172617. In another embodiment, the compound is NSC287296. In another embodiment, the compound is NSC303565. In another embodiment, the compound is NSC316164. In another embodiment, the compound is NSC619165. In another embodiment, the compound is NSC623112. In another embodiment, the compound is NSC631706. In another embodiment, the compound is NSC633406. In another embodiment, the compound is NSC643051, or their combination in other embodiment.

In one embodiment, described herein is a method of inducing apoptosis, or cell-cycle arrest, or both in a p53-deficient tumor cell, comprising the step of contacting the p53-deficient tumor cell with a compound capable of inducing expression of p21, KILLER/DR5, Bax, Bak, Bid, Puma, Noxa, Bnip3L, Bnip3, PIDD, Fas/APO1, caspase 8, caspase 9, caspase 10, caspase 3, caspase 6, caspase 7, APAF1, Smac/DIABLO, cytochrome c, FADD, TRAIL, Fas ligand, Bim, DR4 or their combination.

In another embodiment, the cell cycle arrest or apoptosis is impaired due to p53 deficiency. There are two major classes of cell cycle regulation events: DNA damage events and dependency events. DNA damage events delay cell cycle transitions from G₁ to S and from G₂ to M, thereby providing more time for DNA repair. Essential components of the G₁ checkpoint include ATM, p53, RB, Chk2, and p21^{waf1} (a downstream target of p53). DNA damage activates ATM kinase, which phosphorylates p53 and Chk2, leading to the induction and activation of p53. In turn, p53 transactivates p21^{waf1}, which inhibits the G₁ cyclin-dependent kinases that normally inactivate RB, and thereby represses the E2F transcription factors that initiate S phase. In one embodiment, damage to cellular DNA initiates increased expression of p53 which leads to arrest of the cell cycle. The interruption permits DNA repair to occur before the cell resumes the cell cycle and normal cell proliferation. If repair of the DNA is not successful, the cell then undergoes apoptotic cell death. In another embodiment, when p53 mutates, DNA damaged cells are not arrested in G1 and DNA repair does not take place. The failure to arrest DNA-damaged cells is repeated in subsequent cell cycles permitting and contributes to tumor formation and cancer. The gene encoding p53 is mutated in more than half of all human tumors, suggesting that inactivation of the function of the p53 protein is critical for tumor development.

The N-terminus of p53 (residues 1-90 of the wild-type p53 sequence set forth in SEQ ID NO: 1) encodes its transcription activation domain, also known as transactivation domain. The sequence-specific DNA binding domain has been mapped to amino acid residues 90-289 of wild-type p53. C-terminal to the DNA binding domain, p53 contains a tetramerization domain. This domain maps to residues 322-355 of p53. Through the action of this domain p53 forms homotetramers and maintains its tetrameric stoichiometry even when bound to DNA.

The p53-inducible p21^{WAF1/CIP1} gene encodes a protein which binds to and inhibits a broad range of cycling cyclin-dependent kinase complexes, which promote cell cycle progression. Thus, the consequence of p21^{WAF1/CIP1} activity in one embodiment is growth arrest, which is evident in another embodiment, following exposure of cells to DNA-damaging agents such as γ radiation or adriamycin. In one embodiment, DNA damage brings about p21^{wAF1/CIP1}-induced growth arrest via transcriptional upregulation of p21^{WAF1/CIP1} by the p53 tumor suppressor gene. In one embodiment, p53 deficient cells exposed to γ radiation fail to exhibit either induction of p21^{wAF1/CIP1} expression or G₁ arrest.

In one embodiment, the apoptosis, cell-cycle arrest or both are effected without suppressing the S-phase population of the cell. In one embodiment, the compound capable of inducing apoptosis, or cell-cycle arrest, or both in a p53-deficient tumor cell without suppressing the S-phase population of the cell is NSC5159. In another embodiment, the compound capable of inducing apoptosis, or cell-cycle arrest, or both in a p53-deficient tumor cell without suppressing the S-phase population of the cell is NSC143491. In another embodiment, the compound capable of inducing apoptosis, or cell-cycle arrest, or both in a p53-deficient tumor cell without suppressing the S-phase population of the cell is NSC162908. In another embodiment, the compound capable of inducing apoptosis, or cell-cycle arrest, or both in a p53-deficient tumor cell without suppressing the S-phase population of the cell is NSC254681, or their combination in other embodiments.

In one embodiment, provided herein is a method of increasing p73 transcription in a p53-deficient tumor cell, comprising the step of contacting the p53-deficient tumor cell with a compound that is NSC105900, NSC143491, NSC254681, NSC150412, NSC127133, or their combination.

In another embodiment, provided herein is a method of inhibiting a p53-deficient adenocarcinoma in a subject, comprising the step of administering to the subject a therapeutically effective amount of a composition comprising a compound capable of activating p53-responsive transcriptional activity thereby inducing apoptosis, cell-cycle arrest or both in the p53-deficient tumor cell. In another embodiment, the compound capable of activating p53-responsive transcriptional activity thereby inducing apoptosis, cell-cycle arrest or both in the p53-deficient tumor cell is NSC5159, NSC143491, NSC254681, or their combination.

In one embodiment, the compounds used in the compositions of the methods described herein is TRAIL. In one embodiment, the apoptosis inducing agent is TRAIL, referring to a membrane-bound cytokine molecule that belongs to the family of tumor necrosis factor (TNF). In one embodiment, TRAIL binds with five different receptor molecules, such as DR4, DR5, DcR1, DcR2, and osteoprotegerin (OPG). These receptor molecules, members of the TNF receptor (TNF-R) family, are type I transmembrane polypeptides with 2-5 cysteine-rich domains (CRD) at the extracellular region. DR4 and DR5 containing a cytoplasmic death domain, that is essential for death signaling, are able to transmit apoptosis-inducing activity of TRAIL across the cell membrane.

Four homologous, distinct, human TRAIL receptors exist in one embodiment. In another embodiment two TRAIL-R1TRAIL-R2 having the ability to initiate the apoptosis signaling cascade after ligation and in another embodiment, two others; TRAIL-R3 and TRAIL-R4 lacking the ability to initiate apoptosis signaling cascade after ligation. TRAIL-R3 and TRAIL-R4 have are protective receptors in one embodiment, either by acting as “decoy” receptors or via transduction of an anti-apoptotic signal.

The participation of TRAIL-R3 and -R4 in regulating TRAIL sensitivity may be greater, in one embodiment, in normal cells/tissues or primary tumors than in established tumor cell lines. In another embodiment TRAIL-R3 is a key regulator of the sensitivity of normal cells to TRAIL-induced death, but the addition of cycloheximide may inhibit the production of some other protein (such as FLIP in one embodiment) critical for TRAIL resistance.

In another embodiment, the p53-deficient tumor cell is a colon tumor. In another embodiment, the p53-deficient tumor cell is a small intestine tumor. In another embodiment, the p53-deficient tumor cell is a stomach tumor. In another embodiment, the p53-deficient tumor cell is a liver tumor. In another embodiment, the p53-deficient tumor cell is a kidney tumor. In another embodiment, the p53-deficient tumor cell is a lung tumor. In another embodiment, the p53-deficient tumor cell is a skin tumor. In another embodiment, the p53-deficient tumor cell is a brain tumor. In another embodiment, the p53-deficient tumor cell is a breast tumor. In another embodiment, the p53-deficient tumor cell is a prostate tumor. In another embodiment, the p53-deficient tumor cell is a lymph node tumor. In another embodiment, the p53-deficient tumor cell is a lympoid tumor. In another embodiment, the p53-deficient tumor cell is a thymus tumor. In another embodiment, the p53-deficient tumor cell is an adrenal tumor. In another embodiment, the p53-deficient tumor cell is a thyroid tumor. In another embodiment, the p53-deficient tumor cell is an osteosarcoma. In another embodiment, the p53-deficient tumor cell is a bladder tumor. In another embodiment, the p53-deficient tumor cell is an ovary tumor. In another embodiment, the p53-deficient tumor cell is a uterus tumor. In another embodiment, the p53-deficient tumor cell is a bone tumor. In another embodiment, the p53-deficient tumor cell is a colon adenosarcoma.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### High-Throughout Screening

Cell-based screening for p53-family transcriptional activators was performed using non-invasive bioluminescence imaging to evaluate drug effects. SW480 human colon cancer cells, stably expressing a p53 reporter, PG13-luc, were seeded in 96-well black plate with clear bottom (Corning Inc., Corning, N.Y.) at a density of 5×10⁴ cells/well. Compounds were added to the well at concentrations of 10 μM and 50 μM respectively. p53 transcriptional activity was imaged using an IVIS imaging system (Xenogen Corporation) during a time period ranging from 12-72 hours.

### Western Blotting

Cells were collected and protein concentration was quantified by the Bio-Rad protein assay prior to SDS-PAGE. Proteins were transferred to a PVDF membrane (Immobilon-P, Millipore Corporation, Bedford, Mass.) by a semi-dry transfer apparatus (Bio-Rad Laboratories, Hercules, Calif.). The membranes with transferred proteins were blotted with 10% W/V non-fat dry milk and then incubated with the primary antibody and subsequently secondary antibodies, which were labelled by horseradish peroxidase, or infrared dyes (IR). Signals were visualized by either ECL (Amersham Pharmacia Biotech, England, UK) and exposed to an X-ray film, or scanned by the Odyssey Infared Imaging System (LI-COR Biosciences, Lincoln, Nebr.). Anti-p53, DO-1, was from Santa Cruz biotechnology, Inc. (Santa Cruz, Calif.), anti-p73 (AB-1) and anti-p21 (AB-1) were obtained from Calbiochem (San Diego, Calif.). Anti-ser20 of p53 Cell were obtained from Signaling Technology (Danvers, Mass.) and anti-DR5 antibody was obtained from Cayman Chemical (Ann Arbor, Mich.).

### Flow Cytometry Assay

Adherent cells in a 6-well plate were trypsinized and collected in 15 ml centrifuge tubes to which were added the originally floating cells. The collected cells were ethanol-fixed and stained with propidium iodide (Sigma, St. Louis, Mo.). The DNA content of the stained cells was then measured using an Epics Elite flow cytometer (Beckman-Coulter, Fullerton, Calif.).

si-TAp73 Retrovirus Construction

pBS/U6 vector containing TAp73 RNAi were kindly provided by Leif W. Ellisen (20), Harvard Medical School, from which the expression cassette was removed and recombined to pSIREN-RetroQ (Clontech Laboratories, Inc. Mountain View, Calif.), which was reconstructed to express a blasticidin-resistant marker.

### In Vivo Anti-Tumor Assay

Balb/c nude mice (Charles River Laboratories, Wilmington, Mass.) were inoculated subcutaneously with 2 million HCT116/p53(−/−) cells in an equal volume of Matrigel. When tumor masses reached about 3-5 mm in diameter, mice were treated with the compounds alone by intraperitoneal injection or following a single intravenous dose of TRAIL at 100 μg/mouse. At 7 days after treatment, mice were sacrificed and the tumor masses were weighed. DLD1/PG13 cells were inoculated subcutaneously with 5 million cells. At 24 hours later mice were treated with selected compounds, and subsequently bioluminescence imaging was carried out after 16 hours as previously described [Wang, W. & El-Deiry, W. S. (2003) Cancer Biol Ther 2, 196-202].

### Example 1

### p53 Family Transcriptional Activators Identified from Screening the Diversity Set of the NCI Developmental Therapeutics Program by Bioluminescence Imaging of Human Colon Cancer Cells Expressing Mutant p53 and a p53-Responsive Reporter

A human p53 reporter, PG-13-luc was stably expressed, which carries the firefly luciferase gene under the control of 13 p53-responsive elements, in the human colon adenocarcinoma cell line SW480 that bears a mutant p53 (R273H, P309S). With the firefly luciferase-expressing cell line and by the method of non-invasive real-time imaging [Wang, W. & El-Deiry, W. S. (2003) Cancer Biol Ther 2, 196-202], the National Cancer Institute Developmental Therapeutics Program's (NCI DTP, U.S.) diversity set of approximately 2000 chemical agents accumulated over a 30-year period were screened to identify small molecules that can reactivate p53 signaling in the tumor cells with mutant p53 and cause cell death. The diversity set was initially screened at two doses (10 μM and 50 μM) to discover candidates that can modulate mutant p53, stimulate p73 or induce reporter expression in a manner independent of the p53 family.

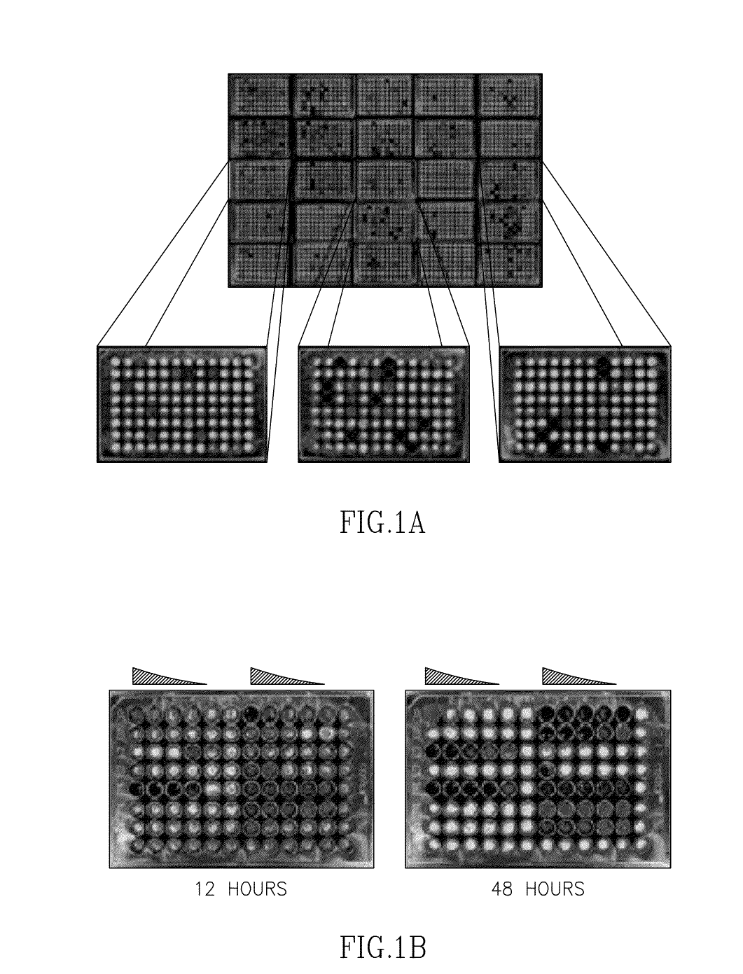

The initial screen (FIG. 1A) manifested two classes of compounds, those that activated the p53-responsive reporter expression without apparent induction of cell death (red color due to high levels of bioluminescence) and those that appeared to cause toxicity and elimination of the baseline reporter signal indicative of cell death (black color due to loss of cell viability), during a time course of 12 to 48 hrs. The two classes of compounds comprised approximately 10% of the total number of compounds tested. It is possible that some compounds leading to apparent loss of cell viability may have inhibited luciferase activity without causing cell death, and these were excluded in secondary screening and not further pursued. Identification of small molecules was sought, which activated a p53 transcriptional activity and subsequently led to cell death. In secondary screening, drug doses were varied over a wider range (from 1 to 200 μM) and time courses were performed to evaluate the fate of cells that showed increased bioluminescence intensity at early time points (within 12 hours) and then loss of viability during a time course of up to 72 hours. Using this secondary screening procedure 33 compounds that appeared to induce p53-responsive reporter activation at low drug doses were identified, but which at later time points or at higher drug doses cell death occurred (FIG. 1B).

### Example 2

### Induction of p53 Target Gene Expression, Cell Cycle Arrest and Apoptosis in p53-Deficient Cells

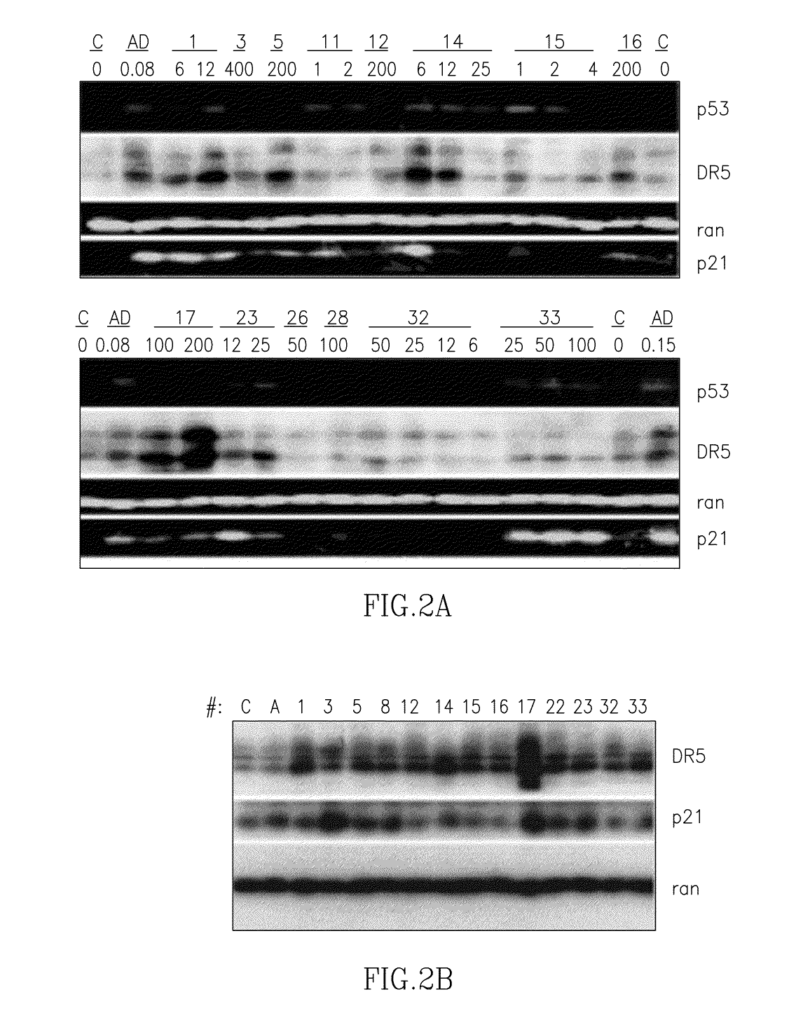

The chemical library screening was directed at restoring “p53 responses” in p53-deficient cells. The small molecules identified by the cell-based screening procedure appeared to be able to restore p53 responses in p53-deficient colon tumors and to eliminate viable cells. Their function was further tested on wild-type p53-expressing and p53-knockout HCT116 colon adenocarcinoma cell lines. A number of candidate modulators of signaling by the p53 family appeared to induce expression of p53 target genes such as p21 or DR5(13) either with or without stabilizing p53 protein in HCT116 cells (FIG. 2A). Compounds #1 (NSC#5159), #14 (NSC#143491), #23 (NSC#254681), and #33 (NSC#639174) appeared to increase p53 expression in parental HCT116 cells and this was accompanied by increased expression of DR5 and p21 proteins (FIG. 2A) in a manner similar to doxorubicin (adriamycin). #11 (NSC#123111) and #15 (NSC#146109) also increased p53 expression but their induction of the p53 targets DR5 and p21 was more modest (FIG. 2A). A number of other compounds including #3 (NSC#28992), #5 (NSC#49692), #12 (NSC#127133), #16 (NSC#150412), and #17 (NSC#162908) appeared to increase p53 target gene expression with a slight or no significant effect on p53 protein expression in HCT116 cells (FIG. 2A).

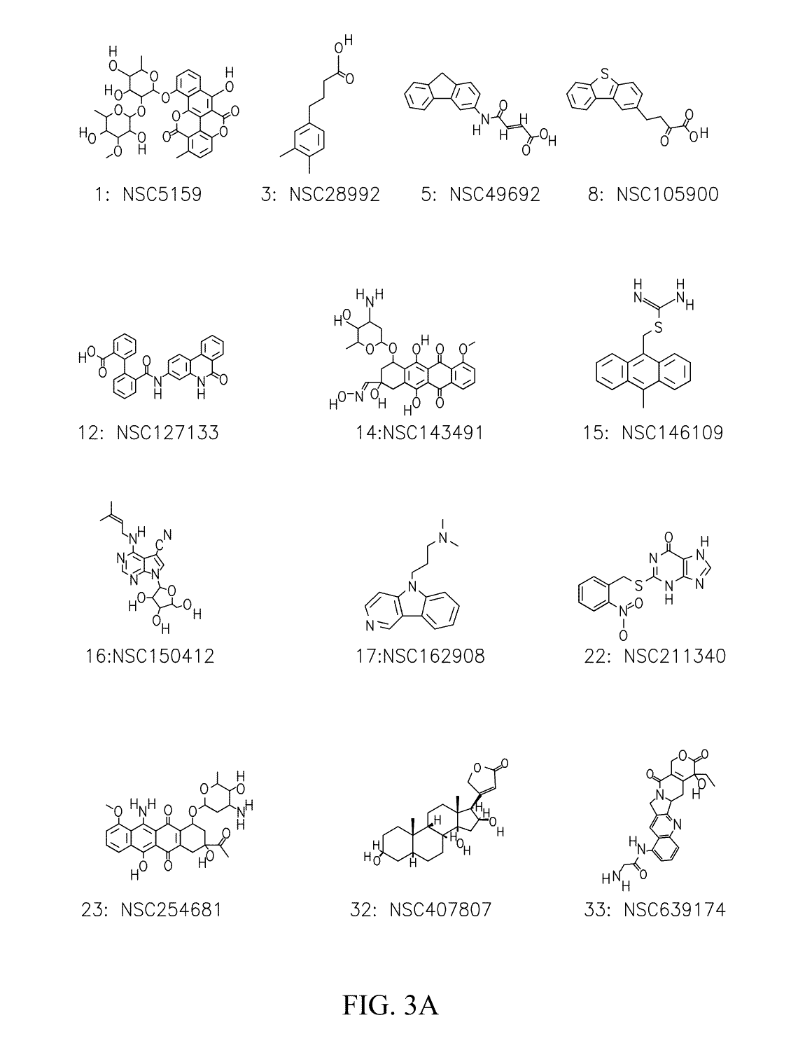

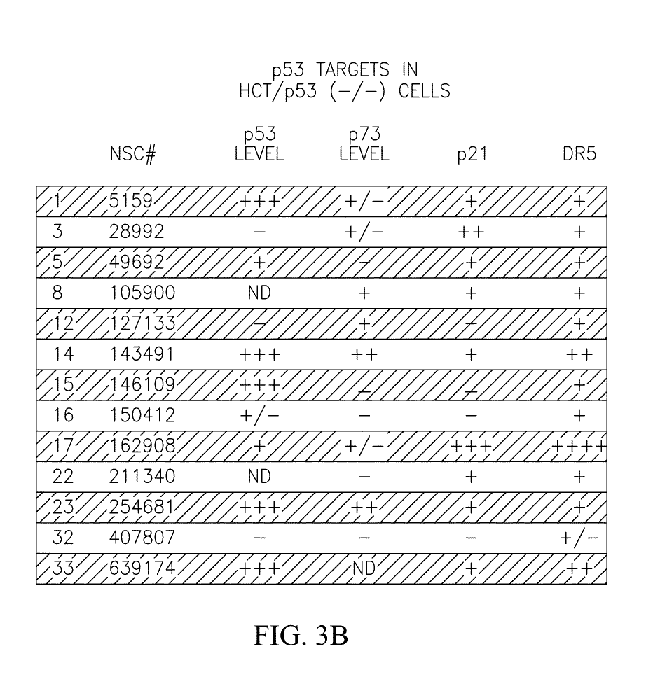

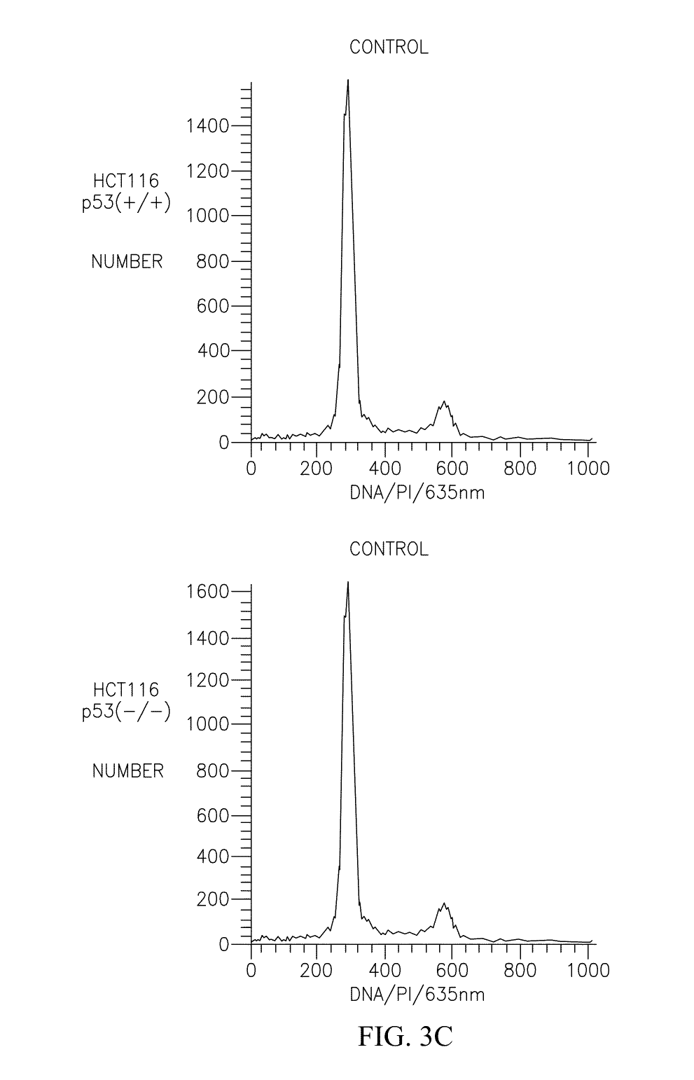

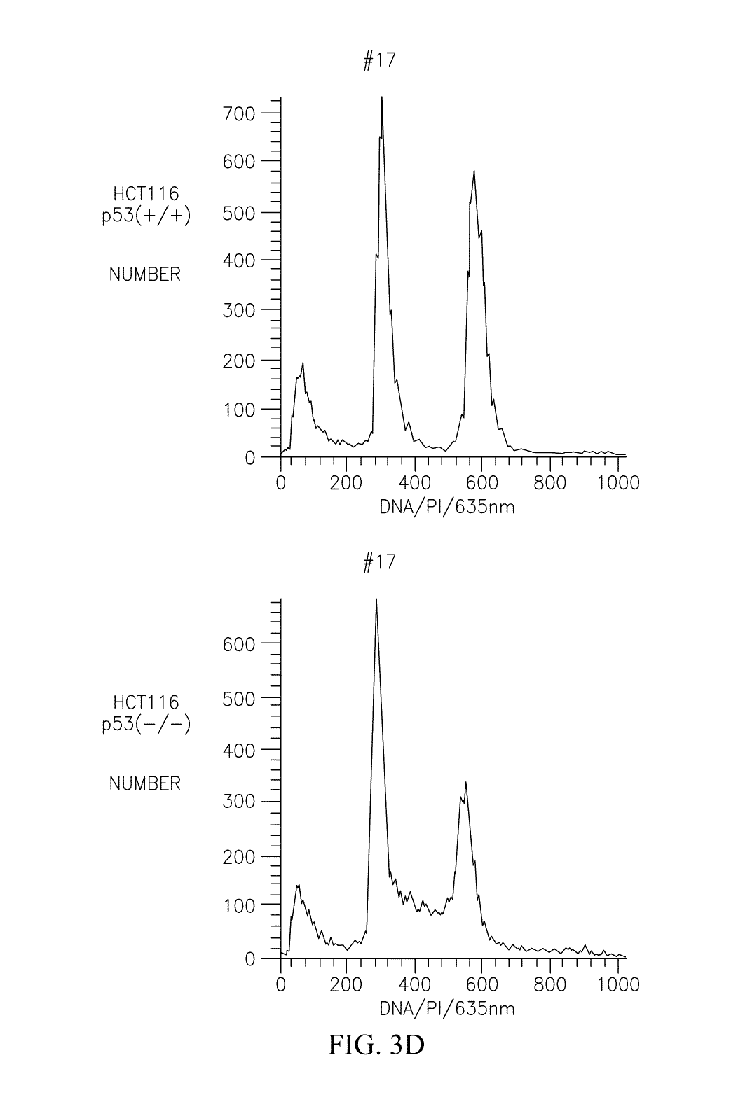

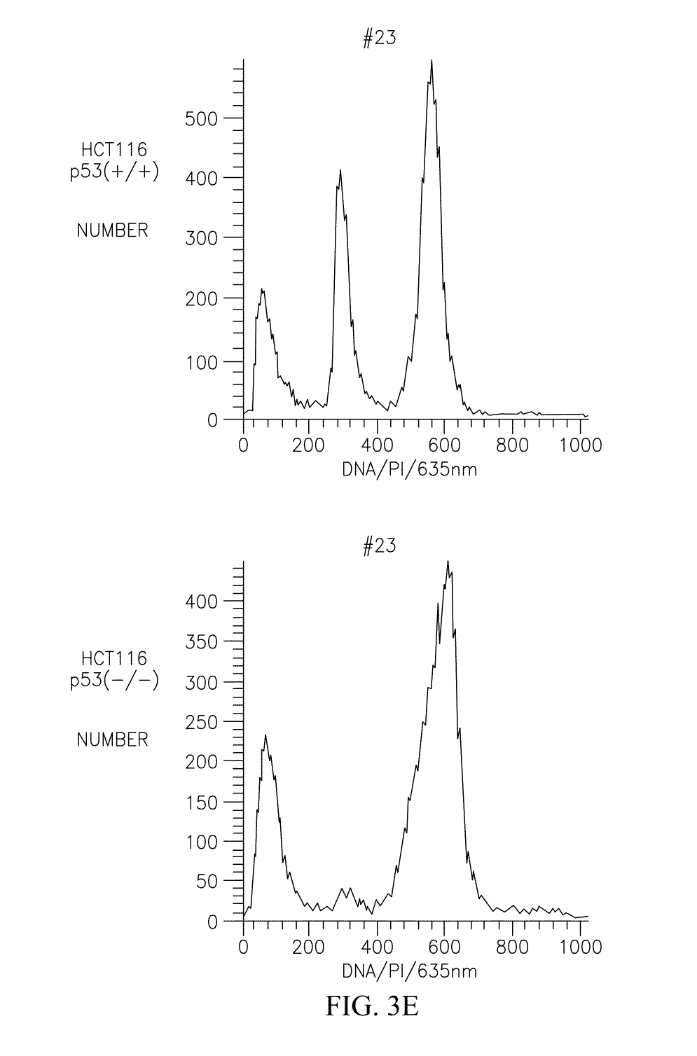

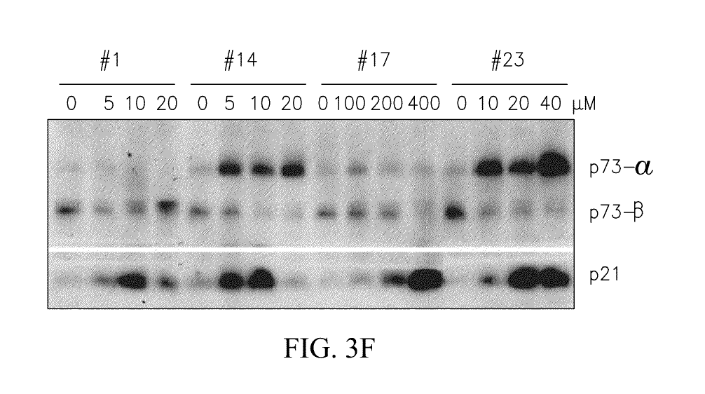

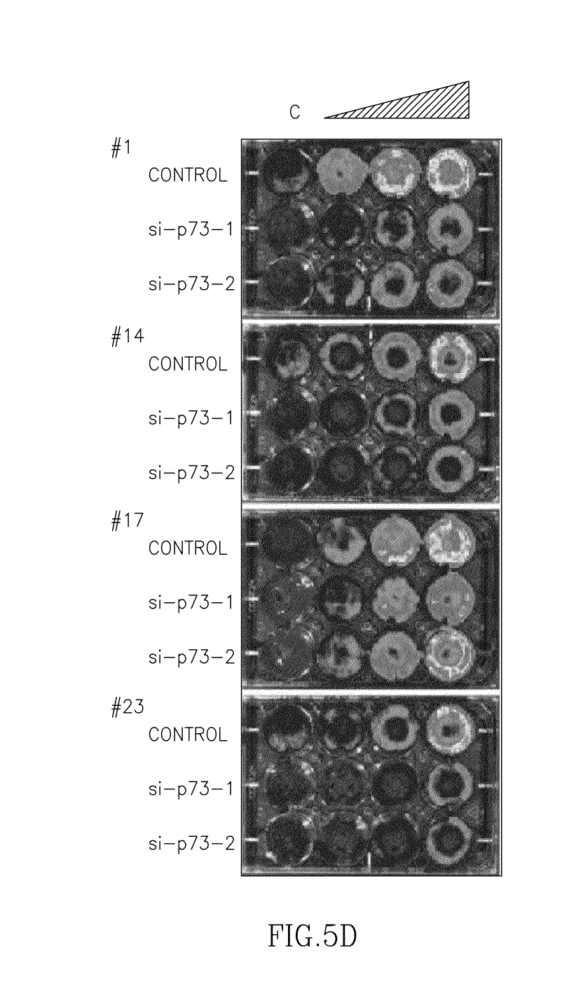

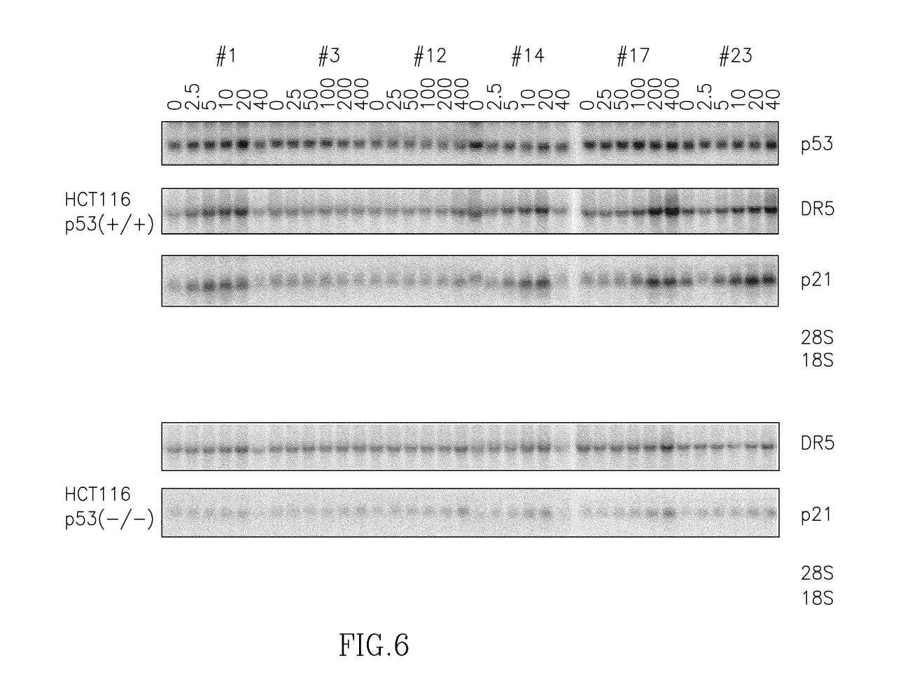

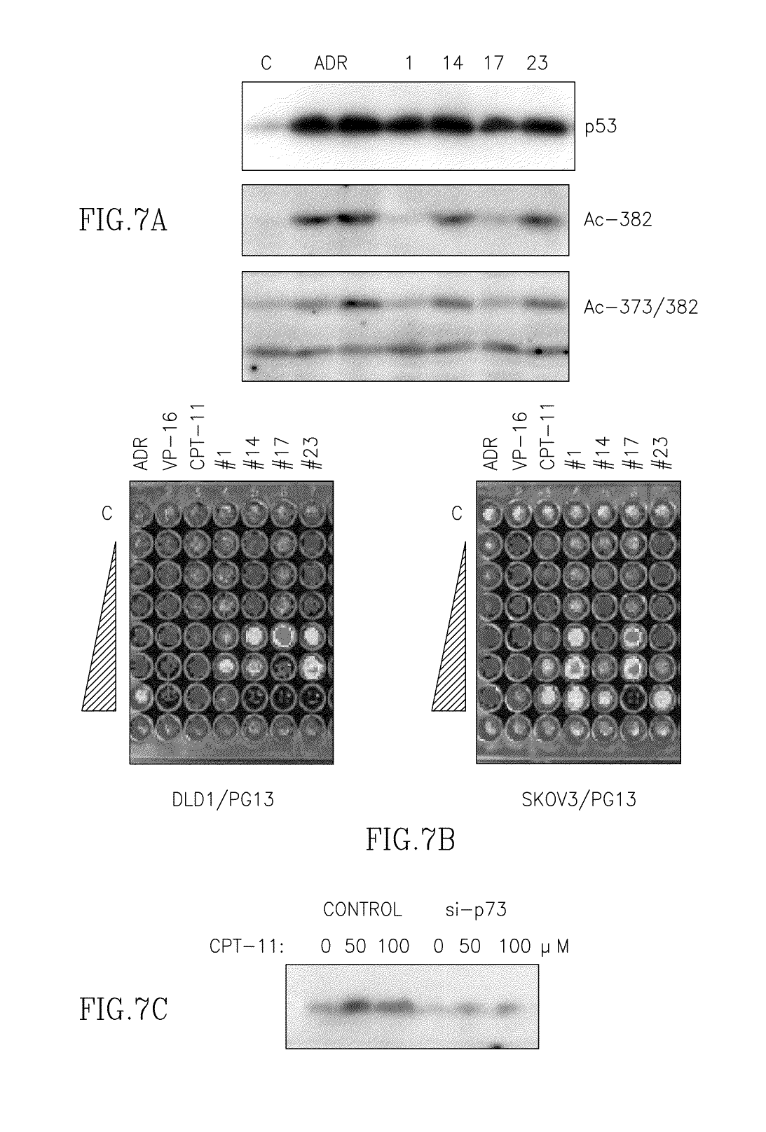

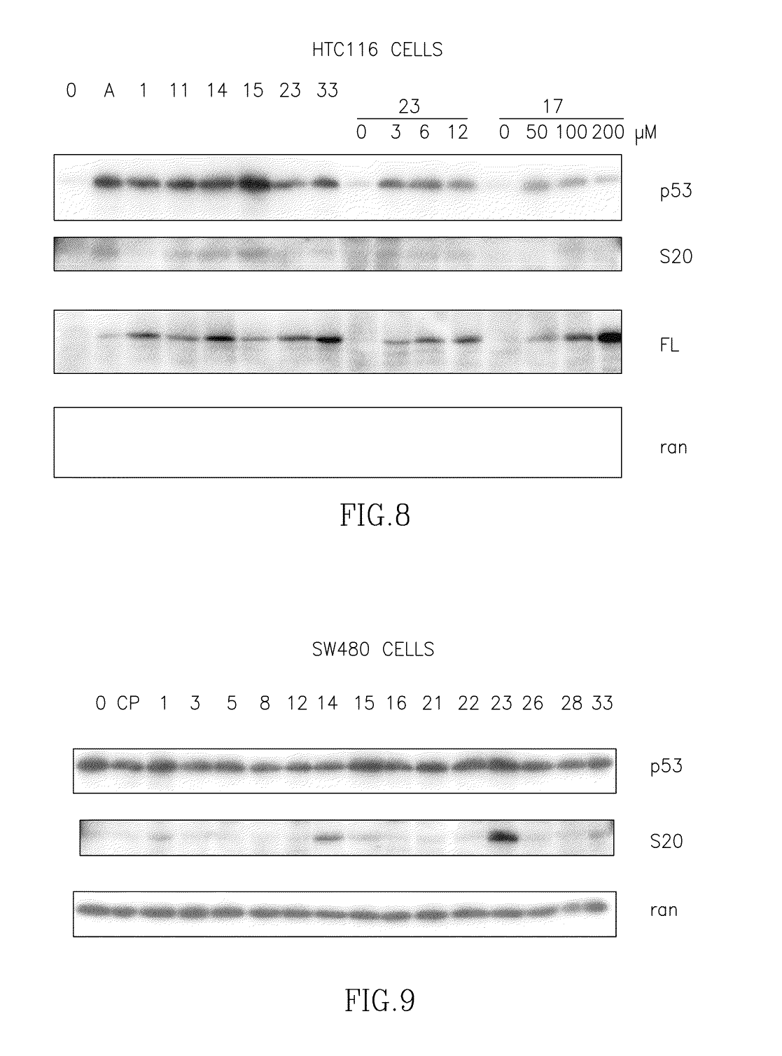

A number of selected compounds was further tested on HCT116/p53^{-/-} cells to verify the possibility of induction of p53 target gene expression in the absence of p53. FIG. 2B shows that the selected compounds appeared to significantly induce DR5 and p21 expression in p53-null HCT116 cells (FIG. 2B), whereas adriamycin had no obvious effect on DR5 and little effect on p21 expression in HCT116/p53^{-/-} cells. The corresponding elevation of mRNA levels of DR5 and p21 (FIG. 7) indicates that some of these compounds activated p53 target gene transcription in both p53^{+/+} and p53^{-/-} cells. Among these compounds, #1, #14, #15, #23, and #33 significantly increased p53 protein levels, while others did not, including #3, #5, #12, #16 (FIG. 2A). Of particular interest, #17 induced the highest p53 transcriptional activity and DR5 levels in both HCT116/p53^{+/+} and HCT116/p53^{-/-} cells (FIG. 2, FIGS. 6B and 8), but modestly induced p53 levels (FIG. 6B) and did not increase p73 expression (FIG. 3F). Moreover, a number of additional compounds tested, including #8 (NSC#105900), #22 (NSC#211340), and #32 (NSC#407807), were found to increase DR5 and p21 expression in the p53-null HCT116 cells (FIG. 2B). The importance of this observation is in establishing that it is possible to identify small molecules with the potential to induce p53 target gene expression in p53-deficient cells.

The ability of selected compounds from the chemical library screen to induce apoptosis of human colon tumor cells and the dependence of their effects on endogenous p53 status was further evaluated. Compounds #1, #14, #17, #23 were chosen because they gave stronger responses in the reporter assays in p53-null HCT116 cells (FIG. 8) in addition to increasing expression of the p53 target genes DR5 and p21 (FIG. 2). These four compounds were found to induced a sub-G1 peak characteristic of apoptosis in either HCT116/p53^{+/+} or HCT116/p53^{-/-} cells (FIGS. 3C-3E). Interestingly, compound #17 induced apoptosis in the p53-null cells without suppressing the S-phase population as observed in the wild-type p53-expressing HCT116 cells. Compound #23 also induced apoptosis in p53-null HCT116 with a greatly reduced G1 arrest as observed in wild-type p53-expressing HCT116 cells (FIGS. 3C-3E). These results show that the cell cycle arrest responses following exposure to either #17 or #23 depended on p53 whereas the apoptotic responses were independent of p53.

### Example 3

### DNA Damage Signaling and p73 are Involved in the Mechanism of Action of Selected Compounds

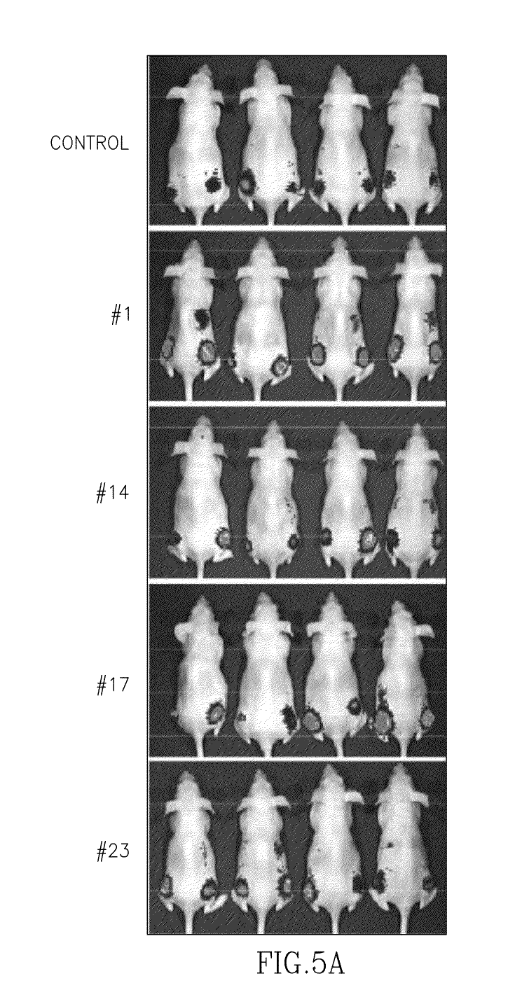

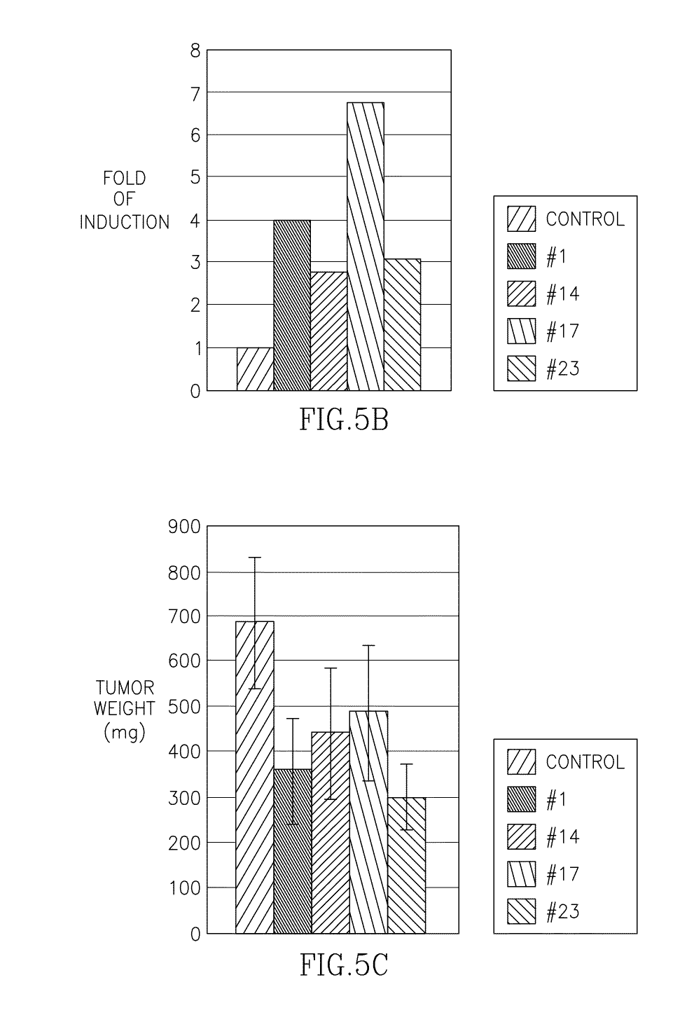

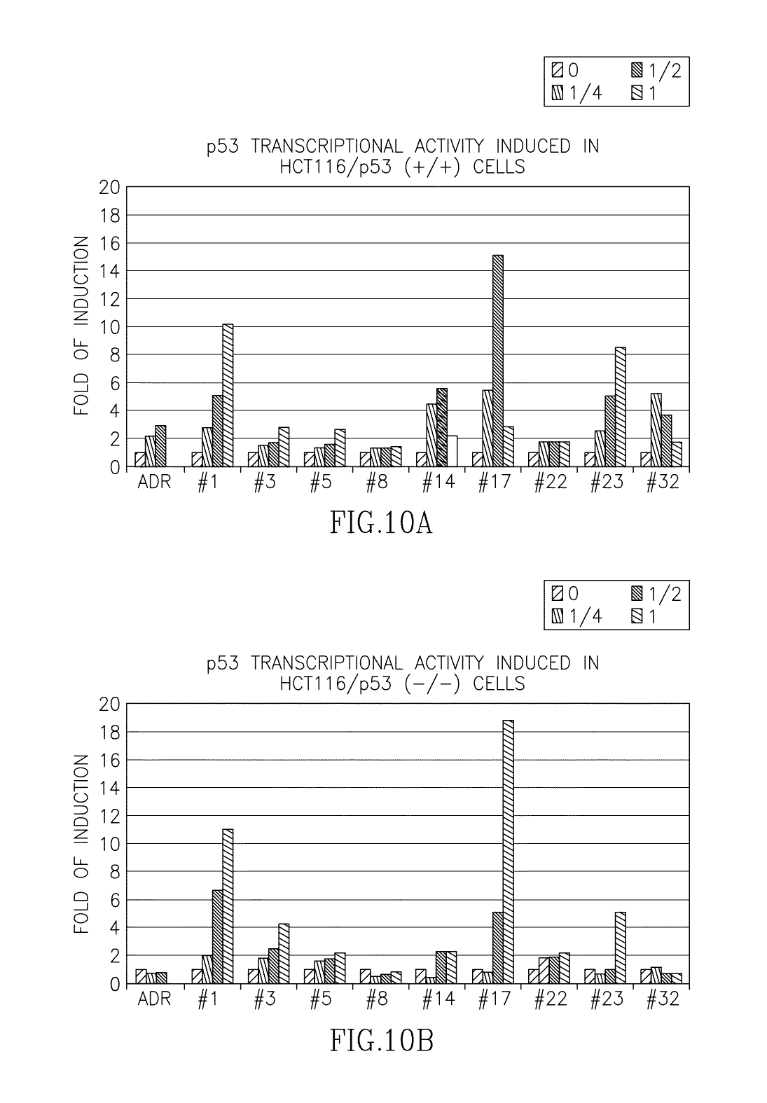

The questioned whether the p53 family member p73 is involved in the p53-responsive transcriptional activity induced by the compounds identified was then tested. p63, the other p53 family member, was not tested, because the TA form of p63 is rarely expressed in malignant and normal tissues except for germ cells of the ovary and testis. As shown in FIG. 3F, #14 and #23 were strong inducers of p73, while the DNA-damaging agent, adriamycin, only increased p73 slightly. Additional compounds including #8, #12, #16 were shown to induce p73 protein expression (data not shown). Knockdown of p73 by retrovirus mediated si-p73 in HCT116/p53^{-/-} cells reduced the baseline expression of the p53 reporter and suppressed p53-responsive transcriptional activity-induced by compounds #1, #14, #23, while the activity induced by #17 was not hindered (FIG. 5D). This indicates that #17 may induce p53 transcriptional activity in p53^{-/-} cells through an alternative pathway that may not involve p73. Knockdown of p73 was demonstrated by western blot (FIG. 10).

In order to determine whether DNA damage signaling is involved in the mechanism of action of selected compounds, Western blot was used to test the status of phosphorylation and acetylation of p53, which are sensitive indicators of DNA damage. It was found that #14 and #23 were strong inducers of p53 phosphorylation at ser20 (FIG. 6B, 6C) and acetylation at lys382 (FIG. 6A). γH2AX was also tested, which was positive after treatment with #14 and #23, but not with #1 and #17. These data indicate that DNA damage signaling is involved in #14 and #23-induced cell death, but not for #1 and #17, which may act by a novel mechanism that requires further investigation.

### Example 4

### In Vivo Anti-Tumor Effects of Selected Compounds

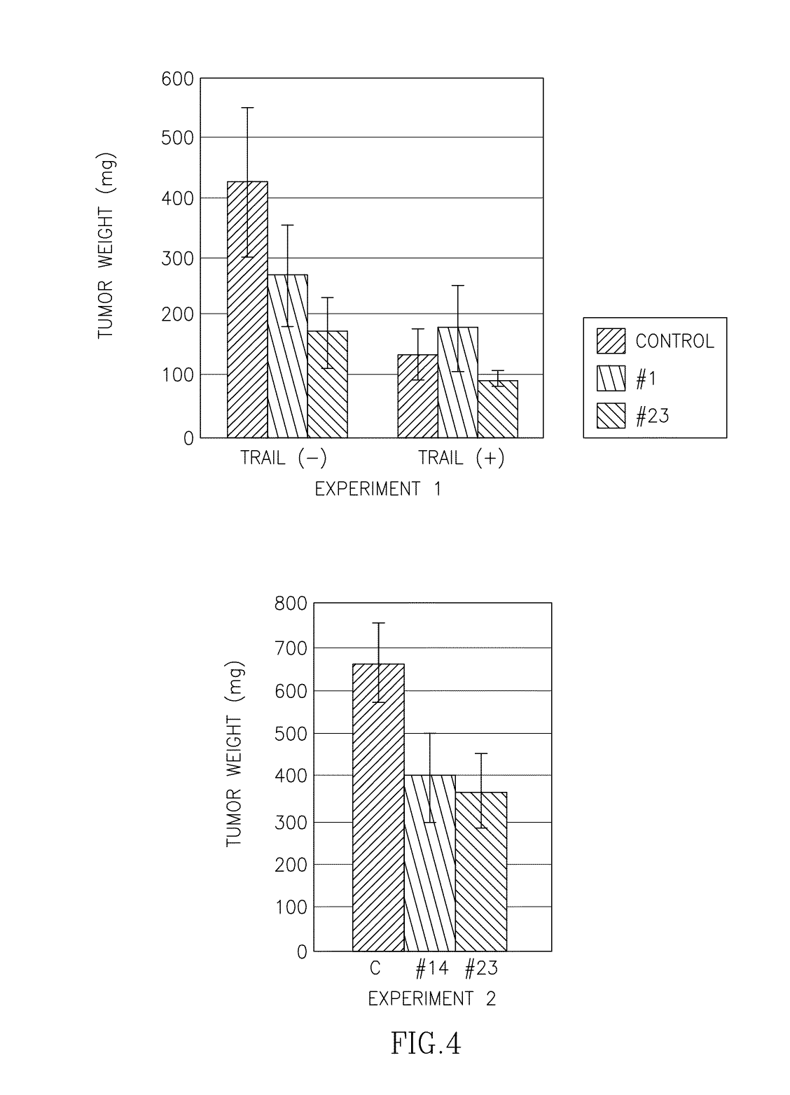

Compounds #1, #14, #17, and #23 were tested in colon-tumor xenograft-bearing mice in order to evaluate their toxicities and potential anti-tumor effects (FIG. 4). These compounds were chosen for further testing based on their ability to strongly induce p53 target gene expression (DR5 and p21) in p53-null cells (FIG. 2B). The initial doses were chosen below maximal tolerated doses based on the NCI DTP toxicology databases for chemical compound testing in vivo so that mice would survive drug administration and allow subsequent evaluation of anti-tumor effects. p53-null HCT116 xenografts were first tested to document anti-tumor effects in p53-deficient tumors and an experiment to simulate therapy of established tumors was designed. A total of 2×10⁶ p53-null HCT116 cells were implanted on opposite flanks subcutaneously in each of 6 nude mice in each group. When tumor masses grew to about 3-5 mm in diameter, drugs were administered intra-peritoneally (#1: 100 mg/kg; #14: 50 mg/kg; #23: 10 mg/kg), and on the next day additional groups received intravenous TRAIL (15) (100 μg/mice via the tail vein). Tumor weights were determined at 7 days later. As shown in FIG. 4, anti-tumor effects were observed with compounds #1, #14, and #23 and a modest additive effect was observed with the combination of #23 with TRAIL. No overt toxicities were observed in mice treated with compounds #1, #14, or #23. Moreover at doses just below the MTD, #17 had no apparent in vivo anti-tumor effect on established HCT116/p53^{-/-} xenograft and at higher doses #17 was found to be toxic to mice. Nonetheless in the future it may be possible to modify the structure of #17 or identify doses where synergistic interactions with TRAIL may be observed.

The question of whether these compounds could stimulate a p53-responsive transcriptional activity in tumor xenografts was tested further. DLD1/PG13 cells were inoculated at the both flanks at a dose of 5 million cells per site. 24 hours after injection, compounds were delivered, and 16 hours later, the intensity of bioluminescence of the tumor cells was imaged and recorded according to protocol previously described [Wang, W. & El-Deiry, W. S. (2003) Cancer Biol Ther 2, 196-202]. All of the four compounds stimulated a p53-responsive transcriptional activity in the tumor xenografts (FIG. 5A, B). Consequently, treatment with the compounds hindered tumor growth (FIG. 5C).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1.-36. (canceled)

37. A method of inhibiting a p53-deficient tumor cell in a subject,
comprising the step of administering to the subject a therapeutically effective amount of a composition comprising a compound capable of activating p53-responsive transcriptional activity thereby inducing apoptosis, cell-cycle arrest or both in the p53-deficient tumor cell, wherein the compound is a compound of table I, or is a compound selected from NSC146505, NSC160470, NSC160471, NSC172617, NSC287296, NSC303565, NSC316164, NSC619165, NSC623112. NSC631706, NSC633406, NSC643051, or a combination thereof.

38. (canceled)

39. (canceled)

40. (canceled)

41. (canceled)

42. (canceled)

43. (canceled)

44. The method of claim 37, whereby the tumor is a colon tumor, a small intestine tumor, a stomach tumor, a liver tumor, a kidney tumor, a lung tumor, a skin tumor, a brain tumor, a breast tumor, a prostate tumor, a lymph node tumor, a lympoid tumor, a thymus tumor, an adrenal tumor, a thyroid tumor, an osteosarcoma, a bladder tumor, an ovary tumor, a uterus tumor, or a bone tumor.

45.-56. (canceled)

57. The method of claim 44, wherein the tumor is a colon tumor.

58. A composition comprising a compound of table I, or a compound selected from NSC146505, NSC160470, NSC160471, NSC172617, NSC287296, NSC303565, NSC316164, NSC619165, NSC623112. NSC631706, NSC633406, NSC643051, or a combination thereof in a therapeutically effective amount to inhibit. a p53-deficient tumor cell in a subject.

59. The composition of claim 58, wherein the tumor is a colon tumor, a small intestine tumor, a stomach tumor, a liver tumor, a kidney tumor, a lung tumor, a skin tumor, a brain tumor, a breast tumor, a prostate tumor, a lymph node tumor, a lympoid tumor, a thymus tumor, an adrenal tumor, a thyroid tumor, an osteosarcoma, a bladder tumor, an ovary tumor, a uterus tumor, or a bone tumor.

60. The composition of claim 59, wherein the tumor is a colon tumor.


SEQUENCE LISTING (2 sequences)

SEQ ID NO: 1 - protein, 393 aa, Homo sapiens [sequence omitted]

SEQ ID NO: 2 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga